(19) United States
(12) Patent Application Publication
(10) Pub. No.: US 2014/0189899 A1
(43) Pub. Date: Jul. 3, 2014

(54) Methods of producing new types of hybrid silk and fibers using insects, animals, and plants

(71) Applicant: Sadia "Ross" Barrameda, Discovery Bay, CA (US)

(72) Inventor: Sadia "Ross" Barrameda, Discovery Bay, CA (US)

(21) Appl. No.: 13/694,762

(22) Filed: Jan. 3, 2013

(51) Int. Cl. A01K 67/04

(52) U.S. Cl. CPC A01K 67/04
USPC 800/21; 435/455; 435/461; 800/278; 435/459

(57) ABSTRACT

The present invention provides a new and improved method for producing hybrid silk and like fibers. The invention provides a method of sequence of use which has many advantages over prior art.

### BACKGROUND OF THE INVENTION

(1) Field of the Invention

The closest cross reference found is Class: For DNA Extraction Methods=435/6.12; 435/6.1; 435/6.11; 536/24.3; 536/24.33; for Hybrid natural fiber composites=428/294.7; 52/793.1; 52/576-577-793; 428/292.1

Current International Class: For DNA Extraction=C12Q 1/68,2006.01/C12P 19/34,2006.01/C07H 21/04,2006.01/for Hybrid natural fiber composites=B32B 13/02,2006.01/E04C 2/34,2006.01/D04H 1/00,2006.01

Field of Search: For DNA Extraction Methods none shown in the search, for Hybrid natural fiber composites=428/117-292.1; 294.4, 297.7; 52/576, 577,793.1; 52/793.11

The field of this invention has to do with the extraction of DNA & RNA from a hemp plant and Omega 3 from fish into animals and other plants producing higher quality silk and or fibers and higher quality meat for human consumption.

(2) Description of the art including information disclosed under 37 CFR 1.97 & 1.98 There are no drawings accompanying this application. The art is described within the descriptions and claims of this application.

### BRIEF SUMMARY OF THE INVENTION

The present invention provides a new and improved method for producing hybrid silk and like fibers. The invention provides a method of sequence of use which has many advantages over prior art.

To attain this, the present invention involves the developing and producing of hybrid silk through bioreactors and hybrid silk worms that when introduced to eating hemp produce new types of silk and fibers. This method uses existing silk worms, hemp plants as a basis as well as other types of caterpillars, insects, including spiders, animals and bioreactors to produce new types of strains of fibers. It is intended that all byproducts are including but not limited to the seeds and eggs that result.

The end result is new combinations of DNA, in both plants and animals, using grafting, crossbreeding, splicing, genetic engineering, bioengineering and using all known and future methods and techniques creating new, different and improved, stronger, more attractive, usable fabrics, materials, fibers, dietary supplements, meats, for the textile, fashion, materials, fiber, food and seed industries.

The process isolates and extracts the DNA from Salmon and other omega 3 producing fish and animals and fusing same into the DNA, genome of chickens, goats and other animals. Isolating and extracting the DNA from the genes of insects and animals causing them to ingest and digest hemp plants and seeds so chickens and other animal will digest (and retain more omega 3 and other beneficial elements) in greater volume. The hemp plants and seeds create a richer omega 3 meat in their meat.

### BRIEF DESCRIPTION OF THE SEVERAL VIEWS OF THE DRAWINGS

There are no drawings as a part of this utility application

### DETAILED DESCRIPTION OF THE PREFERRED EMBODIMENT

There is no preferred embodiment as there are no drawings as a part of this application.

### DETAILED DESCRIPTION OF THE INVENTION

The present invention has a sequence of use comprising, in combination:

Method #1:

Slowly add hemp to the diet of silk worms until they can digest between 1% to 99% of their diet in the various types of hemp, industrial hemp, including, excluding, but not limited to cannabis, marijuana.

Method #2:

Infuse the DNA AND/OR RNA of insects and rodents that normally eat hemp into that of the silk worm to add to and/or increase hemp fibers in the silk. The present invention refers to these new fibers as HEMP SILK™ and SILKY HEMP™.

Method #3:

Infuse the DNA AND/OR RNA of silk worms into insects that eat hemp such as other caterpillars that produce cocoons.

Method #4:

Graft and/or genetically alter mulberry into and/or with hemp and vice versa to make the new species of plants more digestible to the silk worm and other caterpillars to produce new stains of silk, and new types of silk producing worms. The present invention refers to this new plant as HEMPLEBERRY™ and BERRY HEMP™. Genetically infuse hemp DNA into cotton and vice versa to create a new plant. The present invention refers to this new plant as COTTON HEMP™ and HEMPCOTTON™.

Method #5:

Grow various and new types of silk and cotton fibers in a lab utilizing the bio-reactor methods combining the DNA/RNA of the various types of hemp, cotton and silk worms, including but not limited to all plant types that produce fiber, i.e. cotton, hemp, that can be used to make clothing and/or fibers with that of the silk worm and other caterpillars, insects, spiders, rodents and animals.

Method #6:

Insert the genes of silkworms and silk spiders (and other spiders) into hemp and cotton plants in order to produce new fibers in those plants.

Method #7:

Isolate and extract the DNA from Salmon and other omega 3 producing fish and animals and fusing same into the DNA, genome of chickens, goats and other animals. Isolating and extracting the DNA, genes of insects and animals that cause same to ingest and digest hemp and hemp seeds to create riche omega 3 meats in their bodies. The present invention refers to these new meats as “HEMP CHICKEN™, CHICKEN HEMP™, HEMPENCHICKEN™, “HEMP-FED CHICKEN™ and/or OMEGA 3 CHICKEN™ and applies these terms to all animals suitable for human consumption.

The following describes various methods and techniques for infusing the DNA of certain insects, animals and plants into other plants, animals and insects. Those methods may be used in all of these new combinations already described. However, the combinations may be achieved using other known and existing methods and techniques to combine same, and/or techniques and methods that will be discovered in the future. The primary uniqueness in these new ideas combine to create new plants and animals, and not necessarily the techniques and/or methods described below, which are primarily known methods and techniques for combining plants and animal forms of life. Methods #2 and #3 utilize the DNA of the 300 insect pests that cause those insects to ingest hemp and marijuana. Infuse into the DNA AND/OR RNA of silk worms (and other animals and plants mentioned throughout this patent application) the DNA of those pests that causes them to ingest and/or digest hemp, especially the most serious hemp pests which are lepidopterist stem borers, predominately European corn borers (Ostrinia nubilalis) and hemp borers (Graptolitic delineana).

Utilize the below methods to infuse into silk worms (and other animal species) the DNA AND/OR RNA that cause Beetle grubs that bore into stems and roots (e.g., Psylliodes attenuate, Ceutorhynchus rapae, Rhinocus pericarpius, Thyestes gebleri, and several Mordellistena spp.) to ingest and digest hemp.

Utilize the below methods to infuse into the silk worms (and all animal species') DNA AND/OR RNA the DNA/RNA that causes those insects to damage the field crops of leaves and flowering tops caused by caterpillars (e.g., hemp borers and budworms), beetles (e.g., Psylliodes attenuata), bugs, and leaf miners, including the predominant pests in marijuana crops such as the insects with piercing-sucking mouthparts, such as aphids (Pheromone Cannabis, Myzus Persicae, Aphis Fabae), whiteflies (Trialeurodes Vaporariorum, Bemisia spp.), Leafhoppers, and Mealy bugs.

Utilizing the methods described below infused into silkworms, the DNA AND/OR RNA that causes non-insect pests such as mites (Tetranychus Urticae, Aculops Cannabicola) to ingest and digest hemp. Utilize various methods of introducing foreign DNA AND/OR RNA of the hemp eating insects into a eukaryotic cell of the silk worm (and other animals): sometimes relying on physical treatment (Electroporation, Nanoparticles, Magnetofection), other times on chemical materials or biological particles (viruses) that are used as carriers.

UTILIZING CHEMICAL-BASED TRANSFECTION: Chemical-based transfection will be divided into several kinds: cyclodextrin, polymers, liposomes or nanoparticles (with or without chemical or viral functionalization).

Use calcium phosphate, HEPES-buffered saline solution (HeBS) containing phosphate ions combined with a calcium chloride solution containing the DNA AND/OR RNA of the hemp eating insects to transfect the silk worms (and other animals). When the two are combined, a fine precipitate of the positively charged calcium and the negatively charged phosphate will form, binding the DNA AND/OR RNA to be transfected on its surface. The suspension of the precipitate is then added to the cells to be transfected (usually a cell culture grown in a monolayer). The cells of the silk worm (and other animals) take up some of the precipitate, and with it, the DNA AND/OR RNA.

In order to develop new silk (and all animal) producing species, use highly branched organic compounds, so-called dendrimers to bind the DNA AND/OR RNA and get it into the cell of the silk worm (and all other animals).

Utilize the inclusion of the DNA AND/OR RNA to be transfected in liposome's, small, membrane-bounded bodies that are in some ways similar to the structure of a cell and can actually fuse with the cell membrane, releasing the DNA AND/OR RNA of the hemp eating insect into the cells of the silk worm (and all other animals). For eukaryotic cells, transfection is better achieved using cationic liposome's (or mixtures), because the cells are more sensitive.

Use cationic polymers such as DEAE-dextran or, polyethlyenimine. The negatively charged DNA AND/OR RNA of the hemp eating insects binds to the polycation of the silk worm (and all animal species) and the complex is taken up by the cell of the silk worm (and all animal species) via endocythosis.

Non Chemical Methods:

Utilize the electroporation method incorporating an instrument affecting the viability of many cell types creating micro-sized holes transiently in the plasma membrane of cells of the silk worm (and all animal species) under an electric discharge. Similarly, utilize sono-poration via transfection applying sonic forces to the silk worm (and all animal species) cells.

Utilize the optical transfection method where a tiny (˜1 μm diameter) hole is transiently generated in the plasma membrane of the silk worms' cells (and the cells of all animals) using a highly focused laser. In this technique, one cell at a time is treated, making it particularly useful for single cell analysis.

Utilize the gene electro transfer technique enabling transfer of genetic material into the prokaryotic or eukaryotic cells of the silk worm. Basing this method on electroporation, a physical method where transient increases in the permeability of silk worm cell membrane are achieved when submitted to short and intense electric pulses.

Utilize the impalefection method of introducing hemp eating insect DNA AND/OR RNA bound to a surface of a nanofiber that is inserted into a cell of the silk worm (and all animal species). This approach can also be implemented with arrays of nanofibers that are introduced into large numbers of cells and intact tissue of the silk worm (and all animal species).

Utilize the hydrodynamic delivery of the DNA AND/OR RNA of hemp eating rodents most often in their plasmids, including transposons into the silk worm (and all animal species) antimicrobial peptides in the fat body (the insect equivalent of the vertebrate liver) using hydrodynamic injection that involves infusion of a relatively large volume in the blood in less than 10 seconds.

Particle-Based Methods:

Utilize the direct approach to transfection using the “gene gun” where the hemp eating insect's DNA AND/OR RNA is coupled to a nanoparticles of an inert solid (commonly gold) which is then “shot” directly into the silk worm cell's (and all animal species) nucleus.

Utilize the magnetofection, or magnet assisted transfection, a transfection method, which will use magnetic force to deliver the hemp eating insect DNA AND/OR RNA into target cells of the silk worm (and all animal species). Nucleic acids are first associated with magnetic nanoparticles, then the application of magnetic force drives the nucleic acid particle complexes of the hemp eating insets towards and into the target cells of the silk worm (and all animal species), where the cargo is released.

Utilize impalefection, carrying this method out by impaling silk worm (and all animal species) cells by elongated nanostructures and arrays of such nanostructures such as carbon nanofibers or silicon nanowires which have been functionalized with the plasmid DNA AND/OR RNA of the hemp eating insects.

Viral Methods:

Introduce the DNA AND/OR RNA of hemp eating insects into silk worm (and all animal species) cells using viruses as a carrier. In such cases, the technique is called viral transcution, and the cells are said to be transduced. This can be done using insect cells.

Utilize methods of transfection which include nucleofection, heat shock to infuse the DNA AND/OR RNA of hemp eating insects (and other hemp eating pests) into the cells of silk worms (and all animal species).

Stable and Transsient Transfection:

For many of our applications of silk worm (and all animal species) transfection, it is sufficient if the transfected genetic material is only transiently expressed. Since the DNA AND/OR RNA introduced in the transfection process is usually not integrated into the nuclear genome, the foreign DNA AND/OR RNA will be diluted through mitosis or degraded in the silk worm (and all animal species). When it is desired that the transfected gene actually remains in the genome of the cell and its daughter cells, a stable transfection may occur. To accomplish this, a marker gene is contransfected, which gives the cell some selectable advantage, such as resistance towards a certain toxin. Some of the transfected cells will, by chance, have integrated the foreign genetic material into their genome. If the toxin is then added to the cell culture, only those few cells with the marker gene integrated into their genomes will be able to proliferate, while other cells will die. After applying this selective stress (selection pressure) for some time, only the cells with a stable transfection remain and can be cultivated further into the silk worm (and all animal species).

Utilize geneticin as an agent for selecting stable transfection into the silk worm (and all animal species), also known as G418, which is a toxin that can be neutralized by the production of the neomycin resistance gene.

RNA Transfection:

Utilize transfected RNA into silk worm (and all animal species) cells to transiently express its coded protein or to study RNA decay kinetics. The later application is referred as RNA transfection or RNA silencing, and has become a major application in research (to replace the “knock-down” experiments, to study the expression of proteins, i.e. of Endothelin-1) with potential applications in gene-therapy.

Utilizing the embedding of hemp protein [genes] sequenced within silkworm's silk (and all animal species' omega 3) [gene] sequences, causing those proteins to co-assemble into composite fibers in various increasing amounts of hemp protein (omega 3 for animals) starting at 1% up to 99%.

A small increase of hemp genes into the silkworm (and omega 3 in the animals), the hybrid silk, (the hybrid meat) is tougher than natural silk (and omega 3 richer than natural meat. To create these “hemp silkworms,” and “omega 3 rich animals” insert DNA from a hemp (or a type of hemp or cotton) protein into silkworm eggs (and insert DNA from an omega 3 protein, into the embryonic cells of the various animals). To make sure the genetic transformation is successful; add green fluorescent protein to the eggs, (and embryonic cells) so that successfully engineered silkworms (and animals) will glow under blue light.

Utilize all of the above methods to infuse the DNA or silk and web spinning spiders into that of the silk worm and other cocoon spinning caterpillars.

Method 4:

Utilize the transgenic method to have genes of hemp inserted into mulberry plant and vice versa. In many cases the inserted DNA of hemp and mulberry has to be modified slightly in order to correctly and efficiently express in the host mulberry organism and vice versa.

Utilize Cisgenic method to crossbreed by conventional means.

Utilize “knock out” method to knock out the dominant gene of mulberry to allow phenotype to develop and vice versa. Another strategy is to attach the gene of hemp to a strong biological “promoter” of mulberry or other elements and vice versa to see what happens when it is over expressed. Utilize the common technique to find out where the hemp gene and mulberry genes are expressed by attaching them to a “GUS reporter system” or a similar “reporter gene” that allows visualization of the location. Utilize RNA technology to match the inserted DNA of an endogenous gene already in the plant. When the inserted gene is expressed it can repress the “translation” of the endogenous protein. Utilize host delivered RNA systems so the plants will express RNA that will not interfere with silk worms (and all animal species) that have been engineered to have the appetite of the insects, nematodes and other parasites protein synthesis. This provides an additional novel way of enduring, the genetically engineered silk worms (and all animal species) to the genetically engineered hemp/mulberry plants.

Utilize Mutagenesis to change the genetic information of the hemp and mulberry organisms in a stable manner, resulting in mutations. The results will occur both spontaneously and as a result of exposure to mutagens. This will also be achieve experimentally using laboratory procedures. Also utilize the following techniques of spontaneous hydrolysis, cross linking, dimerization, and intercalation between bases, backbone damage, insertional mutagenesis, and intentional error in replication, mutagenesis as a laboratory technique, random mutagenesis and site-directed mutagenesis, combinatorial mutagenesis, insertional mutagenesis, and other types of mutagenesis.

Utilize molecular cloning to create recombinant DNA of all of the above combinations including polymerase chain reaction (PCR) used to direct the replication of any specific DNA sequence chosen by patent applicant. The present applicant herein may from time to time form recombinant DNA of the above mention using a cloning vector, a DNA molecule that will replicate within a living cell. Vectors will generally be derived from plasmids or viruses, and represent relatively small segments of DNA that contain necessary genetic signals for replication, as well as additional elements for convenience in inserting foreign DNA, identifying cells that contain recombinant DNA, and, where appropriate, expressing the foreign DNA. The choice when choosing to utilize vector for molecular cloning depends on the choice of host organism, the size of the DNA to be cloned, and whether and how the foreign DNA is to be expressed. The DNA segments will sometimes be combined by using a variety of methods, such as restriction enzyme/lipase cloning or Gibson assembly. The present applicant will normally utilize standard cloning protocols, the cloning of any DNA fragment essentially involving seven steps: (1) Choice of host organism and cloning vector, (2) Preparation of vector DNA, (3) Preparation of DNA to be cloned, (4) Creation of recombinant DNA, (5) Introduction of recombinant DNA into the host organism, (6) Selection of organisms containing recombinant DNA, (7) Screening for clones with desired DNA inserts and biological properties. Patent applicant will sometimes utilize molecular cloning to achieve commercialization of the ideas recorded throughout.

Method 5:

Create in a lab, using the bioreactor and/or the protein6 reactor techniques, attached to an artificial vine, or other apparatus, man-made silkworms that imitate the processes of spinning a cocoon so that as long as it is artificially fed with hemp and/or mulberry and/or any elements that will give man made silkworms such capacity to produce silk indefinitely, both of traditional consistency and of new improved strains of silk created from the unique combinations listed above throughout. The present invention includes using the same techniques of causing all other animals to consume hemp and transferring the DNA to those animals that will cause them to produce in their meats omega 3, as mentioned herein for chicken, to all consumable animals. It is to be understood that the present invention is not limited in its application to the details of construction and the arrangement of the components set forth in the description. The invention is capable of other embodiments and of being practiced and carried out in various ways, also, it is to be fully understood that the phraseology and terminology employed herein are for the purpose of description and should not be regarded as limited. As such, those skilled in the art will appreciate that the conception, upon which this disclosure is based, may readily be utilized as a basis for the designing of other structures, methods and systems for carrying out the several purpose of the present invention. It is important, therefore, that the claims be regarded as including such equivalent constructions insofar as they do not depart from the spirit and scope of the present invention. Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the present application. The abstract is neither intended to define the invention of the application, which is measured by the claims, nor is it intended to be limited as to the scope of the present invention in any way.

It is therefore an object of the present invention to provide a new and improved method for producing hybrid silk and like fibers which has the advantage of the prior art devises and none of the disadvantages.

It is another object of the present invention to provide a new and improved method for producing hybrid silk and like fibers which may be easily and efficiently manufactured and marketed.

It is a further object of the present invention to provide a new and improved method for producing hybrid silk and like fibers which is of a durable and reliable construction. An even further object of the present invention is to provide a new and improved method for producing hybrid silk and like fibers which is susceptible of a low cost of manufacture with regard to both materials and labor, and which accordingly is then susceptible of low prices of sale to the consuming public, thereby making such a method for producing hybrid silk and like fibers economically available to the buying public. Still yet another object of the present invention is to provide a new and improved method for producing hybrid silk and like fibers which provides in the apparatuses and methods of the prior art some of the advantages thereof, while simultaneously overcoming some of the disadvantages normally associated therewith. Still another object of the present invention is to provide a new and improved method for producing hybrid silk and like fibers operable from a novice's level. Yet another object of the present invention is to provide a new and improved method for producing hybrid silk and like fibers. It is therefore an additional object of the present invention to provide a new and improved method for producing hybrid chickens and other animals fit for human consumption which have the advantage of the prior art devises and none of the disadvantages.

It is another object of the present invention to provide a new and improved method for producing hybrid animals which may be easily and efficiently genetically modified, raised and marketed.

It is a further object of the present invention to provide a new and improved method for producing hybrid animals which produce omega 3 in their bodies to enrich their dietary benefits for human consumption. An even further object of the present invention is to provide a new and improved method for producing hybrid animals which are susceptible of a low cost of production with regard to both materials and labor, and which accordingly is then susceptible of low prices of sale to the consuming public, thereby making such a method for producing hybrid animals and economically available to the buying public.

Still yet another object of the present invention is to provide a new and improved method for producing hybrid animals (both living for the farms and in a lab for bio-reactors) which provide in the apparatuses and methods of the prior art some of the advantages thereof, while simultaneously overcoming some of the disadvantages normally associated therewith.

Still another object of the present invention is to provide a new and improved method for producing hybrid animals operable from a novice's level.

Yet another object of the present invention is to provide a new and improved method for producing hybrid animals. These together with other objects of the present invention, along with the various features of novelty which characterize the present invention, are pointed out with a particularity within the claims annexed in the utility patent.

As to the manner of usage and operation of the present invention, the same should be apparent from the above description. Accordingly, no further discussion relating to the manner of usage and operation will be provided. With respect to the above description then, it is to be realized that the optimum dimensional relationships of the invention, to include variations, materials, function and sequence of operation, are deemed readily apparent and obvious to one skilled in the art and all descriptions in the specification are intended to be encompassed by the present invention.

Therefore, the foregoing is considered a written description of the principles of the invention. Further, since numerous modifications and changes will readily occur it is not desired to limit the invention to the exact construction and operation described, and accordingly, all suitable modifications and equivalents may be resorted to, falling within the scope of the invention.

I claim:

1. (canceled)

2. (canceled)

3. (canceled)

4. (canceled)

5. (canceled)

6. (canceled)

7. (canceled)

8. Process for the genetic transformation of silkworms with DNA and/or RNA of insects or rodents that normally eat hemp comprising of:

9. The composition of claim 1, wherein slowly add hemp to the diet of silk worms until they can digest between 1% to 99% of their diet in the various types of hemp, industrial hemp, including, excluding, but not limited to cannabis, marijuana.

10. The composition of claim 1, wherein infuse the DNA AND/OR RNA of insects and rodents that normally eat hemp into that of the silk worm to add to and/or increase hemp fibers in the silk. The present invention refers to these new fibers as HEMP SILK™ and SILKY HEMP™.

11. The composition of claim 1, wherein infuse the DNA AND/OR RNA of silk worms into insects that eat hemp such as other caterpillars that produce cocoons.
